(19) United States
(12) Patent Application Publication
(10) Pub. No.: US 2007/0251141 A1
(43) Pub. Date: Nov. 1, 2007

(54) Method for Preparation, Use and Separation of Fatty Acid Esters

(75) Inventors: Shailendra Bist, Torrance, CA (US); Bernard Y. Tao, Lafayette, IN (US); Samia A. Mohtar, West Lafayette, IN (US)

Correspondence Address: BAKER & DANIELS LLP, 300 NORTH MERIDIAN STREET, SUITE 2700, INDIANAPOLIS, IN, 46204, US

(73) Assignee: PURDUE RESEARCH FOUNDATION, West Lafayette, IN (US)

(21) Appl. No.: 11/690,540

(22) Filed: Mar. 23, 2007

Related U.S. Application Data
(63) Continuation-in-part of application No. 11/668,865, filed on Jan. 30, 2007.
Application No. 11/668,865 is a continuation-in-part of application No. 11/068,104, filed on Feb. 28, 2005.
(60) Provisional application No. 60/547,992, filed on Feb. 26, 2004.

(51) Int. Cl. C07C 51/43; C10L 1/18

(52) U.S. Cl. 44/385; 554/186

(57) ABSTRACT

A method for treating a fatty acid methyl ester. The method can include mixing the fatty acid methyl ester with an amount of urea and an amount of alcohol to make (i) a urea/fatty acid methyl ester ratio of from about 0.1:1 to 1:1 wt/wt and (ii) an alcohol/fatty acid methyl ester ratio of from about 3:1 to 10:1 wt/wt; heating the fatty acid methyl ester/urea/alcohol mixture to a temperature at which a homogenous mixture is obtained, cooling the fatty acid methyl ester/urea/alcohol mixture to a temperature where a solid phase comprising a clathrate of urea and saturated fatty acid ester and a liquid phase comprising unsaturated fatty acid methyl ester are formed, and separating the solid phase from the liquid phase. The unsaturated fatty acid methyl ester is useful as a fuel resistant to gel formation at low temperature.

### PRIORITY CLAIM

This application claims the benefit of U.S. Continuation-in-Part patent application Ser. No. 11/668,865, filed Jan. 30, 2007, which is a Continuation-in-Part of U.S. patent application Ser. No. 11/068,104, filed Feb. 28, 2005, which claims priority from U.S. Provisional Patent Application Ser. No. 60/547,992 filed Feb. 26, 2004, the complete disclosures of which are hereby expressly incorporated by reference.

### TECHNICAL FIELD OF THE DISCLOSURE

The present invention generally relates to fatty acid esters and a method for preparation and separation of fatty acid esters. The present invention particularly relates to a method for separating saturated and unsaturated fatty acids. Separated fractions of fatty acid esters are useful as renewable fuels.

### BACKGROUND OF THE DISCLOSURE

Urea is known to form inclusion complexes with long chain organic compounds. This was first discovered and reported by F. Bengen in a German patent filed in 1940. Later studies from the late forties to the early fifties reported the selectivity of urea in forming complexes with long chain organic molecules. This selectivity was found to be based on; a) Carbon chain length, b) presence of unsaturation in the molecule, and c) degree of unsaturation. The formation of these complexes was found to be a useful technique for the separation of a mixture of saturated and unsaturated organic compounds, e.g. fractionation of a mixture of free fatty acids. Various techniques for the formation of such complexes were also studied, however with little or no focus on the process parameters. Work done by Hayes et al., on the fractionation of fatty acids studied various process parameters that effect the formation of urea inclusion complexes, the product yields, and the composition of fractions obtained. Patents U.S. Pat. No. 5,106,542, U.S. Pat. No. 5,243,046 describe the art of fractionating fatty acid mixtures via urea inclusion. U.S. Pat. No. 5,679,809 describes the concentration of polyunsaturated fatty acid ethyl esters via urea inclusion.

Fatty acid esters find a variety of uses including in foodstuffs, nutritive compositions, pharmaceuticals, cosmetics, dermatological compositions, and drying oils for coatings and paints.

Biodiesel, according to ASTM D-6751 specification, is defined as a fuel comprised of mono-alkyl esters of long chain fatty acids derived from vegetable oils or animal fats. The alkyl group is of the type CₙH₂ₙ₊₁, preferable methyl (CH₃), the oil source is preferable soybean. Biodiesel derived from a soy oil source is referred to as SME (soy methyl esters) or soy biodiesel elsewhere in the following text.

References:

1. Bengen, F., German Patent Application O. Z. 12438, Mar. 18, 1940.

2. Swem, D. “Urea and Thiourea Complexes in Separating Organic Compounds,” Industrial and Engineering Chemistry, Vol. 47, 216-221, 1955.

3. Swem, D., Parker, W. E., “Application of Urea Complexes in the Purifcation of Fatty Acids, Estes, and Alcohols. 1. Oleic Acid from Inedible Animal Fats,” JAOCS, 431-434, 1952.

4. Newey, H. A., Shokal, E. C., Mueller, A. C., Bradley, T. F., “Industrial and Engineering Chemistry,” Vol. 42, 2538-2540, 1950.

5. Schlenk, H., Holman, R. T., “Separation and Stabilization of Fatty Acids by Urea Complexes,” Journal of American Chemical Society, vol. 72, 5001-5005, 1950.

6. Hayes, D. G., Bengtsson, Y. C., Alstine, J. M. V., Setterwall, F., “Urea Complexation for the Rapid, Ecologically Responsible Fractionation of Fatty Acids from Seed Oil,” vol. 75, JAOCS, 103-1409, 1998.

7. Hayes, D. G., Bengtsson, Y. C., Alstine, J. M. V., Setterwall, F., “Urea-Based Fractionation of Seed Oil Samples Containing Fatty Acids and Acylglycerols of Polyunsaturated and Hydroxy Fatty Acids,” Vol. 77, JAOCS, 207-213, 2000.

8. Hayes, D. G., “Free Fatty Acid Fractionation via Urea Inclusion Compounds,” Vol. 13, INFORM, 832-833, 2002.

9. Hayes, D. G., Alstine, J. M. V., Asplund, A. L., “Triangular Phase Diagrams to Predict the Fractionation of Free Fatty Acid Mixtures via Urea Complex Formation,” Separation Science and Technology, Vol. 36, 45-58, 2001.

10. Lee, L. A. Johnson and E. G. Hammond, “Reducing the Crystallization Temperature of Biodiesel by Winterizing Methyl Soyate,” JAOCS, Vol. 73, No. 5 (1996).

11. R. O. Dunn, M. W. Shockley, and M. O. Bagby, “Improving the Low-Temperature Properties of Alternative Diesel Fuels: Vegetable Oil-Derived Methyl Esters,” JAOCS, Vol. 73, No. 12 (1996).

12. Diks, R. M. M., Lee, M. J., “Production of Very Low Saturate Oil Based on the Specificity of Geotrichum Candidum Lipase,” JAOCS, Vol. 76, No. 4, 1999.

13. Shimada, Y., Maruyama, K., Okazaki, S., Nakamura, M., Sugihara, C., “Enrichment of Polyunsaturated fatty Acids with Geotrichum Candidum Lipase,” JAOCS, Vol. 71, 951-953, 1994.

14. U.S. Pat. No. 5,678,809, “Concentration of Polyunsaturated Fatty Acid Ethyl Esters and Preparation Thereof”.

15. U.S. Pat. No. 5,106,542, “Process for the Continuous Fractionation of a Mixture of Fatty Acids”.

16. U.S. Pat. No. 5,243,046, “Process for the Continuous Fractionation of a Mixture of Fatty Acids”.

17. U.S. Pat. No. 6,444,784 B1, “Wax Crystal Modifiers”.

18. U.S. Pat. No. 6,409,778 B1, “Additive for Biodiesel and Biofuels”.

19. International Publication No. WO 99/62973, “Wax Crystal Modifiers Formed Form Dialkyl Phenyl Fumarate”

20. International Publication No. WO 00/32720, “Winterized Paraffin Crystal Modifiers”

21. U.S. Pat. No. 3,961,916, “Middle Distillate Composition with Improved Filterability and Process Thereof”

22. U.S. Pat. No. 5,726,048, “Mutant of Geotricum Candidum Which Produces Novel Enzyme System to Selectively Hydrolyze Triglycerides”.

23. U.S. Pat. No. 6,537,787, “Enzymatic Methods for Polyunsaturated Fatty Acid Enrichment”

24. U.S. Pat. No. 5,470,741, “Mutant of Geotrichum Candidum Which Produces Novel Enzyme System to Selectively Hydrolyze Triglycerides”

25. Kocherginsky et al., “Mass Transfer of Long Chain Fatty Aids Through Liquid-Liquid Interface Stabilized by Porous Membrane,” Separation Purification Technology, Vol. 20, 197-208, 2000.

26. U.S. Pat. No. 4,542,029, “Process for Separating Fatty Acids”

27. U.S. Pat. No. 4,049,688, “Process for Separating Esters of Fatty Acids by Selective Adsorption”

28. U.S. Pat. No. 4,129,583, “Process for Separating Crystallizable Fractions From Mixtures Thereof

29. Maeda, K., Nomura, Y., Tai K., Uneo, Y., Fukui, K., Hirota, S., “New Crystallization of Fatty Acids From Aqueous Ethanol Solution Combined with Liquid-Liquid Extraction,” Ind. Eng. Chem. Res., Vol. 38, 2428-2433, 1999.

The above references 1.-29. are incorporated herein by reference.

Methods in the art for fractionating SME to improve its cold flow properties are based on thermal crystallization (winterization) followed by filtration (with or without solvent). Both techniques rely on the difference in crystallization temperature of the saturated and unsaturated components of SME. Saturation describes compounds having all available valence bonds of carbon atoms in the compound attached to other atoms. Unsaturation describes compounds in which not all available valence bonds of carbon are satisfied resulting in the formation of double or triple bonds. Carbon-carbon double bonds are the form of unsaturated bonds contemplated in plant origin fatty acid methyl esters. The saturates crystallize at a higher temperature and can be removed via filtration, centrifugation etc. However, due to co-crystallization of the components by the winterization technique, significant unsaturates are also removed, resulting in high losses of the preferred unsaturated fatty acid methyl esters. For a cloud point depression (C.P.) of −16 C. almost 75% of the starting material was removed in work done by Dunn et al. Cloud point describes the temperature at which a waxy solid material appears as a fuel is cooled. These techniques involve cooling to very low temperatures and process time running into days. It would be advantageous to identify a method for separation of fatty acid methyl esters applicable to industrial scale application.

### SUMMARY OF THE DISCLOSURE

This invention relates to the fractionation/separation of fatty acid methyl esters, exemplified SME into saturated fatty acid-rich and unsaturated fatty acid-rich fractions via the use of urea inclusion/urea complexation. Operation of diesel engines using renewable energy sources including triglyceride derived fuels is known, as is the challenge of overcoming negative properties of triglyceride derived fuels, e.g., the gelling of bioderived diesel (biodiesel) at higher temperatures than petroleum derived fuels. The composition of biodiesel (for a typical sample of soy biodiesel) is as given in table (1).

TABLE 1

| Fatty Acid Methyl Ester | % by Weight |
|---|---|
| Methyl Palmitate (C16:0) ¹ | 10.3 |
| Methyl Sterate (C18:0) | 4.7 |
| Methyl Oleate (C18:1) | 22.5 |
| Methyl Linoleate (C18:2) | 54.1 |
| Methyl Linolenate (C18:3) | 8.3 |

¹ The parenthetical reference (C nn:n) indicates first the number of carbon atoms of the molecule: followed by the number of carbon-carbon double bonds in the molecule.

The present invention includes one or more of the following features: A controlled C.P. depression can be achieved ranging from about 2 to about 26 C.°. ‘Cloud point depression’ is the difference in C.P. of the product and the starting material. The process can be optimized for processing cost, processing time, scalability and robustness for a desired C.P. depression. The disclosed process benefits from the opportunity to recycle and reuse raw materials. The process is ecologically friendly with all raw materials, intermediates and final products and wastes being biodegradable. The disclosed process provides an efficient method of obtaining an unsaturate rich fraction and a saturate rich fraction from a mixture of fatty acid methyl esters (FAME), particularly those derived from vegetable source.

### DETAILED DESCRIPTION OF THE DRAWINGS

The aforementioned and other features and objects of this invention, and the manner of attaining them, will become apparent and the invention itself will be better understood by reference to the following description of several embodiments of the invention taken in conjunction with the accompanying drawings, wherein:

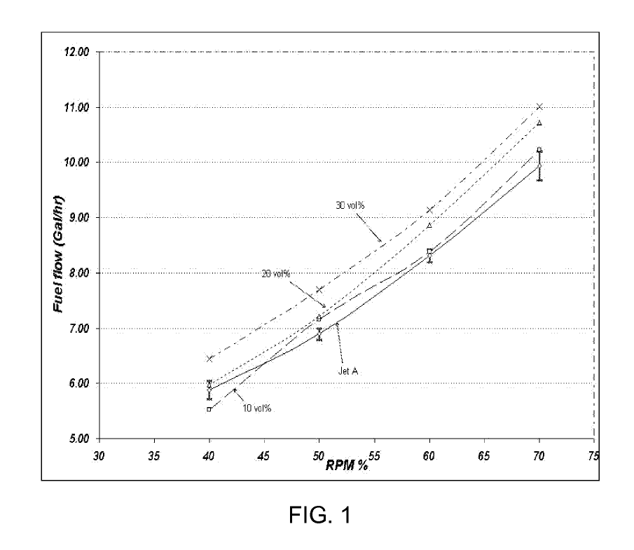

FIG. 1 is a fuel flow chart plotting fuel consumption for a turbine engine power range from 40 to 70% for mineral jet fuel and mineral fuel containing stated soy methyl ester content.

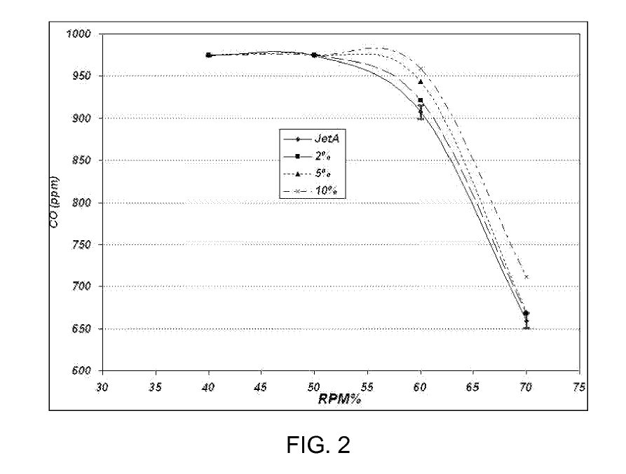

FIG. 2 is a chart of CO production in exhaust gas of a turbine engine for mineral jet fuel and mineral fuel containing stated soy methyl ester content.

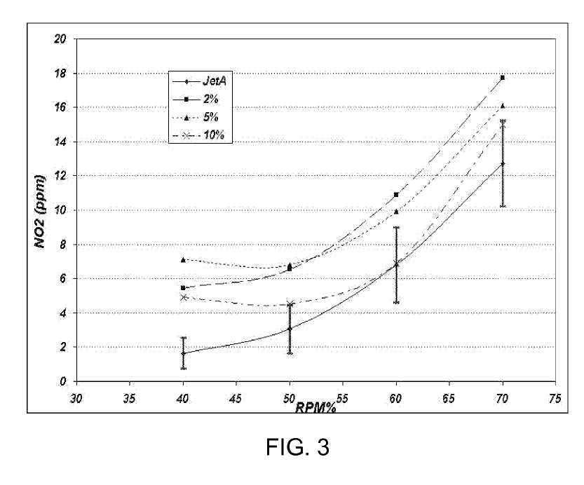

FIG. 3 is a chart of NO₂ production in exhaust gas of a turbine engine for mineral jet fuel and mineral fuel containing stated soy methyl ester content.

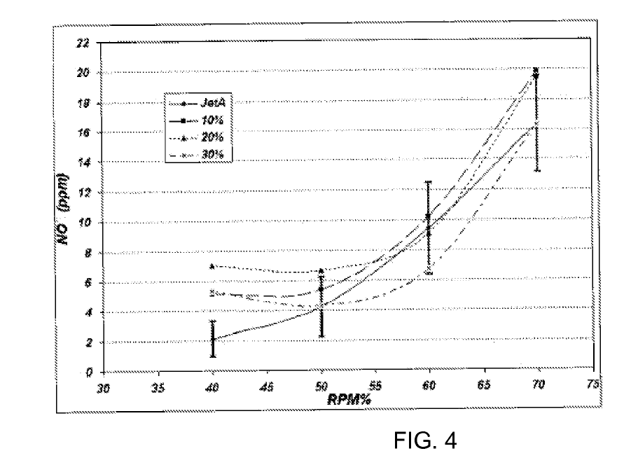

FIG. 4 is a chart of NO production in exhaust gas of a turbine engine for mineral jet fuel and mineral fuel containing stated soy methyl ester content.

### DETAILED DESCRIPTION OF THE DISCLOSURE

While the invention is susceptible to various modifications and alternative forms, specific embodiments are shown by way of examples. It should be understood, however, that there is no intent to limit the invention to the particular form disclosed, but on the contrary, the intention is to cover all modifications, equivalents, and alternatives falling within the spirit and scope of the invention as defined by the appended claims.

SME has proven to be an extender/additive/replacement for diesel fuel, heating oil and studies are on for its development as an aviation turbine fuel extender. A challenge to the utilization of biodiesel is its poor cold flow properties. The total saturate content of about 14-16% (wt/wt) causes the C.P. to be about 0 C.° and pour point to be around −2 to −4 C.°. This limits the use of SME at low temperatures. Various efforts have been made to reduce or depress the C.P. of SME by: 1) removal of saturated components, 2) use of cold flow additives, 3) use of branched chain alcohol esters, 4) combinations thereof.

The popular method for removal of saturate components is winterizing or cold filtering. Various studies have been conducted, however these methods have very low yields for any significant change in the C.P. Cold flow additives have been successful in lowering the P.P (Pour Point), however have little or no effect on the C.P. of SME. Branched chain alcohol esters have poor yields during the esterification reaction, higher raw material cost with only a small depression in C.P.

A C.P. depression ranging from −2 C.° to −60 C.° is disclosed, by a controlled removal of the saturated fatty acid-rich fraction, with unsaturated fatty acid-rich fraction yields ranging from 98%-41% of the starting material, respectively. The process parameters of greater significance being 1) urea/FAME/Alcohol (weight/weight/weight ratio), and 2) the temperature to which the methyl ester clathrate mixture is cooled. The rate of cooling appears to play a lesser role in the formation of urea clathrates and therefore separation of saturated from unsaturated fatty acid methyl ester.

Depending upon the desired C.P. drop (or end-point C.P.), a combination of urea/FAME/Alcohol ratio, and cooled to temperature may be selected to achieve economic commercial completion of the clatherate formation and separation of fatty acid methyl esters. Various such combinations are possible for the same C.P. drop. The urea/FAME ratio may range from 0.1:1 to 1:1 wt/wt. The alcohol/FAME ratio may range from 3:1 to 10:1 wt//wt. Typical methyl ester preparation involves transesterification of fatty acid with methanol in batch vessels at temperatures from 50 to 75° C. Transesterification reactants comprise the fatty acid source such a soy oil, an alcohol and advantageously, a catalyst. Methanol is generally chosen as the reactant of choice for soy oil esterification resulting in formation of the methyl ester from triglyceride. A hydroxide catalyst is generally the commercial choice to accelerate the transesterification although the reaction also responds to acid catalysis. Generally mineral acids or mineral bases are selected as transesterification catalysts.

Typically transesterification of soy fats is considered commercially complete after a reaction time from three to one hours at reaction conditions. Total time of reactants in the reaction vessel may exceed the stated times if it is necessary to heat reactants to reaction temperature in situ. Commercial completion of the transesterification reaction occurs when the economics do not warrant continued maintenance of reaction conditions. Commercial completion may be influenced by many factors such as the equipment involved: its capital cost/depreciation status, its operating expense, its size, its geometry, the separations equipment available, raw material cost, labor cost, or even the time of day as it relates to operator's shift change.

The range of possible fat sources is not limited. Commercial fat sources are generally chosen from oilseeds, often locally produced, such as soybeans and canola. The carbon content of fatty acids from such sources ranges from 16 to 22 carbon atoms per fatty acid molecule.

Raw materials of fats and alcohol are supplied to the reaction vessel in the molar ratio of 1 mole fat (triglyceride) to 3 moles alcohol. Although the process is operable outside this ratio, unreacted raw materials result. The reaction is observed to be nearly stoichiometric although it may be advantageous to add excess alcohol to the esterification step as will be discussed further. One percent catalyst by weight of fat is sufficient facilitate the reaction at a commercially acceptable rate. Insufficient catalyst results in a slowed reaction; excess catalyst is not observed to materially increase the reaction rate and may require additional separation effort at the completion of the reaction.

The transesterification reaction generates glycerin. If allowed a period of quiescence, the glycerin phase will separate from the FAME at the commercial completion of the transesterification. The phases may then be decanted. Other phase separation methods, such as a centrifuge may be used to accelerate and enhance the separation of glycerin from the ester.

The instant method calls for the addition of an alcohol as a urea solvent, such as methanol, and urea to the ester reaction product. By weight, the ratio of urea to FAME reaction product may be in the range from 0.1:1 to 1:1. Urea forms solid phase clathrates with the saturated fatty acid esters.

The addition of sufficient alcohol to dissolve the added urea is suggested. A suitable addition of alcohol results in a ratio of alcohol to the methyl ester from 3:1 to 10:1 wt/wt. Excess alcohol does not noticeably enhance the clathrate formation. Alcohol present after clathrate formation is often separated from the predominate unsaturated ester by methods depending on the relative vapor pressure of the two components, for example: distillation, or flash evaporation. Economic considerations encourage limiting alcohol addition to an amount necessary to dissolve the added urea.

Methanol is preferred for the esterification. Use of methanol as the solvent for urea in preference to C₂-C₄ alcohols eliminates the need to store and handle additional reagents for separation of fatty acid methyl esters. After formation of the FAME and glycerin, a convenient manufacturing sequence separates the glycerin phase from the fatty acid ester phase, followed by addition of urea and alcohol to the FAME. The urea and alcohol may be added separately or as a solution of urea dissolved in alcohol. An option afforded by the use of methanol as urea solvent is the convenient continuation of the process by conducting subsequent clathration step in the same vessel used for the ester formation. By continuing the process in the same vessel with methanol as the solvent, capital investment is reduced by eliminating additional process steps to first remove residual methanol prior to addition of C₂-C₄ alcohols and reduced capital investment for separations vessels and equipment required to provide the reduced cloud point fatty acid methyl ester.

As an alternative to first separation of glycerin from the methyl ester followed by the clathrate formation, sufficient excess methanol may be included at the esterification step to dissolve urea subsequently added to form clathrates of the saturated fatty acid esters. This alternative results in the glycerin (generated from the transesterification process) being present in the methanol phase as clathrates are formed. The liquid phase unsaturated methyl esters, methanol and glycerin may be separated in a single step from the clathrate, solid phase. Followed by subsequent separation of the components.

Dissolution of urea in the methyl ester—alcohol solution proceeds quickly with stirring at temperatures in the range of 50-75 C.°. The rate of heating of the mixture has not been observed to have a material effect on the yield of the product or the C.P. achieved.

It has been observed that the cooling rate has little material influence on the C.P. Yields are impacted more significantly by the final cooled to temperature. Cooled to temperatures of 20 to 25° C. the mixture of saturated fatty acid esters urea clathrates, unsaturated fatty acid methyl esters, excess/unreacted methyl alcohol, excess/unreacted urea and optionally glycerin give good yields and high clathrate formation of saturated esters.

The solid phase including clathrates of the saturated methyl esters may be separated from the liquid phase comprising unsaturated-methyl-esters, methanol, and dissolved urea and optionally glycerin by convenient solid-liquid separation means such as filtration or centrifuge.

If present, glycerin may be decanted from the liquid phase. Alcohol present in the liquid phase rich in unsaturated fatty acid esters may be recovered by evaporation at a temperature between 30-50 C.° (preferably under vacuum). The remaining filtrate is then washed with warm acidic water (60-70 C.°, pH 3-4) to remove urea and alcohol. The water wash may be carried out in steps, washing the filtrate with warm, acidified water in each step, or in a continuously manner. Suitable purity of filtrate may be achieved with two step washes with water volumes equal to the filtrate volume. Continuous washing is successful with 3-4 water volumes.

The saturate rich fraction may be obtained from the raffinate by dissolving and washing with warm acidified water (60-70 C.°, pH 3-4). The warmed saturate rich fraction phase separates from the aqueous phase. The saturate rich fraction has utility such as a hydrocarbon source in chemical manufacturing or an additives to heating oil and other heavy oils where C.P. is not a critical property. Urea can be recovered for re-use by evaporation of the wash water.

The invention/technique is illustrated by the following examples:

### EXAMPLE 1

Soy methyl ester prepared as described is analyzed for composition. The starting soy methyl ester had the composition and properties according to Table 2:

TABLE 2

| | Percentage by weight |
|---|---|
| Fatty Acid Methyl Ester | composition |
| Methyl Palmitate (C16:0) | 9.15 |
| Methyl Stearate (C18:0) | 3.78 |
| Methyl Oleate (C18:1) | 23.52 |
| Methyl Linoleate (C18:2) | 55.25 |
| Methyl Linolenate (C18:3) | 7.64 |
| Others | 0.66 |
| Total Saturates | 12.93 |
| Cloud Point: (C. °) | 0 |

24.057 g of soy methyl ester and 10.077 g of urea were added to 160 mL of ethanol and the mixture was heated to 67 C.°, with constant stirring. A homogenous mixture was obtained with all the urea dissolving at this temperature. The mixture was then cooled at a rate of 1.19 C.°/min to a final temperature of 20 C.°. The urea inclusion compounds (clathrates) formed were separated by filtration. The filtrate was then heated to 30 C.° and 70% of the starting volume of ethanol was recovered via evaporation under vacuum. The remaining filtrate was twice washed with equal volume of water (60 C.°, pH 3). 18.83 g of fractionated soy methyl ester (78.38% by wt of the starting soy methyl ester) was recovered with the composition and properties according to Table 3. Recovered ethanol is available for re-use in the process.

TABLE 3

| | Percentage by weight |
|---|---|
| Fatty Acid Methyl Ester | composition |
| Methyl Palmitate (C16:0) | 6.34 |
| Methyl Stearate (C18:0) | 1.39 |
| Methyl Oleate (C18:1) | 24.57 |
| Methyl Linoleate (C18:2) | 59.61 |
| Methyl Linolenate (C18:3) | 8.07 |
| Others | 0.02 |
| Total Saturates | 7.73 |
| Cloud Point: (C. °) | −10 |

### EXAMPLE 2

24.053 g of soy methyl ester having the composition according to Table 2 and 18.045 g of urea were added to 160 mL of ethanol and the mixture was heated to 73 C.°, with constant stirring. A homogenous mixture was obtained with all the urea dissolving at this temperature. The mixture was then cooled at a rate of 1.19 C.°/min to a final temperature of 20 C.°. The urea inclusion compounds formed were then separated by filtration. The filtrate was then heated to 30 C.° and 52% of the starting volume of ethanol was recovered via evaporation under vacuum. The filtrate was twice washed with equal volume of water (60 C.°, pH 3). 15.97 g of fractionated soy methyl ester (66.39% by wt of the starting soy methyl ester) was recovered with the composition and properties according to Table 4.

TABLE 4

| | Percentage by weight |
|---|---|
| Fatty Acid Methyl Ester | composition |
| Methyl Palmitate (C16:0) | 1.55 |
| Methyl Stearate (C18:0) | 0.00 |
| Methyl Oleate (C18:1) | 21.92 |
| Methyl Linoleate (C18:2) | 69.47 |
| Methyl Linolenate (C18:3) | 7.03 |
| Others | 0.03 |
| Total Saturates | 1.55 |
| Cloud Point: (C. °) | −26 |

### EXAMPLE 3

24.056 g of soy methyl ester having the composition according to Table 2 and 16.041 g of urea were added to 160 mL of ethanol and the mixture was heated to 72 C.°, with constant stirring. A homogenous mixture was obtained with all the urea dissolving at this temperature. The mixture was then cooled at a rate of 1.32 C.°/min to a final temperature of 30 C.°. The urea inclusions compounds formed were then separated by filtration. The filtrate was then heated to 30 C.° and 63% of the starting volume of ethanol was recovered via evaporation under vacuum. The filtrate was twice washed with equal volume of water (60 C.°, pH 3). 18.25 g of fractionated soy methyl ester (75.86% by wt of the starting soy methyl ester) was recovered with the composition and properties according to Table 5.

TABLE 5

| | Percentage by weight |
|---|---|
| Fatty Acid Methyl Ester | composition |
| Methyl Palmitate (C16:0) | 2.25 |
| Methyl Stearate (C18:0) | 0.00 |
| Methyl Oleate (C18:1) | 22.45 |
| Methyl Linoleate (C18:2) | 68.53 |
| Methyl Linolenate (C18:3) | 6.75 |
| Others | 0.02 |
| Total Saturates | 2.25 |
| Cloud Point: (C. °) | −16 |

### EXAMPLE 4

24.089 g of soy methyl ester having the composition according to Table 2 and 16.044 g of urea were added to 160 ml of ethanol and the mixture was heated to 72 C.°, with constant stirring. A homogenous mixture was obtained with all the urea dissolving at this temperature. The mixture was the cooled at a rate of 10.71 C.°/min to a final temperature of 20 C.°. The urea inclusions compounds formed were then separated by filtration. The filtrate was then heated to 30 C.° and 63% of the starting volume of ethanol was recovered via evaporation under vacuum. The filtrate was twice washed with equal volume of water (60 C.°, pH 3). 15.64 g of fractionated soy methyl ester (64.92% by wt of the starting soy methyl ester) was recovered with the composition and properties in Table 6.

TABLE 6

| | Percentage by weight |
|---|---|
| Fatty Acid Methyl Ester | composition |
| Methyl Palmitate (C16:0) | 2.08 |
| Methyl Stearate (C18:0) | 0.00 |
| Methyl Oleate (C18:1) | 24.04 |
| Methyl Linoleate (C18:2) | 66.03 |
| Methyl Linolenate (C18:3) | 7.54 |
| Others | 0.01 |
| Total Saturates | 2.08 |
| Cloud Point: (C. °) | −23 |

### EXAMPLES 5-7

Fuel for turbine engines is specified by ASTM standard D-1655. Plant sourced oils have limited penetration in to the market for turbine fuel.

A commercially sourced soybean oil derived fatty acid methyl ester the properties of which are described in Table 2 was fractionated as described herein. The ‘as obtained’ fraction analysis and the fraction analysis after processing appears in Table 8. The fractionated soy methyl ester of Examples 5-7 was then blended with the Commercial Jet A fuel to yield the properties according to Table 7.

TABLE 7
Turbine Fuel

| Property- | | 9 Parts Jet A: 1 | 7 Parts Jet A: 3 | 9 parts Jet A 1 Part |
|---|---|---|---|---|
| Measurement | ASTM D | Part Fractionated | Parts Fractionated | Fractonated SME - |
| Units | 1655 | SME - Example 5 | SME - Example 6 | Example 7 |
| Density - kg/m³ | 775-840 | 817.8 | 831.4 | 817.8 |
| Viscosity cSt @−20° C. | maximum 8.0 | 5.471 | — | — |
| Freeze Point - ° C. | maximum −40° C. | −42° C. | −41° C. | −40° C. |
| Net Heat of | minimum 42.8 | 42.67 | 41.43 | 42.58 |
| Combustion - MJ/kg | | | | |
| Acid Value - mgKOG/g | maximum 0.01 | 0.016 | 0.028 | 0.016 |

TABLE 8

| | Commercial | Fractionated | Fractionated | Fractionated |
|---|---|---|---|---|
| | Soy Methyl | SME | SME | SME |
| | Ester | Example 5 | Example 6 | Example 7 |
| Component | Percent by Weight | | | |
| methyl | 9.15 | 3.48 | 1.30 | 6.53 |
| palmitate | | | | |
| methyl | 3.78 | 0.23 | 0.10 | 0.54 |
| stearate | | | | |
| methyl | 23.52 | 28.99 | 28.17 | 28.70 |
| oleate | | | | |
| methyl | 55.25 | 58.12 | 60.62 | 55.95 |
| linoleate | | | | |
| methyl | 7.64 | 9.18 | 9.80 | 8.28 |
| linolenate | | | | |
| unknown | 0.66 | 0 | 0 | 0 |

The fractionated soy methyl ester was blended with Jet A fuel in the ratios indicated in Table 7 yielded the properties noted. The blended fuel has demonstrates that the requirements of ASTM D-1655 are attainable with blends including soy methyl ester.

Combustion studies of soy methyl ester blends with commercial Jet A show non-critical deviation from the combustion of commercial Jet A fuel. An Allison stationary 250 turbine having a relatively low compression ration of 6.2:1 was used for the combustion study. FIG. 1 shows the fuel flow rate over a power range from 40 to 70 RPM % for Jet A, and soy methyl ester blends of 10%, 20% and 30% with Jet A.

Controlled emissions for Jet A and soy methyl ester blends are shown in FIG. 2 for carbon monoxide, FIG. 3 for nitrogen dioxide, and FIG. 4 for nitrogen monoxide.

### EXAMPLE 8

Transesterification of soy oil with methanol in a vessel was completed with 3 molar parts methanol to 1 molar part refined soy oil. The liquid components were heated to 65° C. NaOH as a catalyst at the rate of 1% by weight of soy oil was included. The condition was maintained for one hour with continuous mixing. The resulting two phases were separated by decantation. Analysis of the methyl ester phase disclosed the composition by weight in Table 9.

TABLE 9
Soy Oil Methyl Ester

| | % by weight |
|---|---|
| Methyl Palmitate (C16:0) ¹ | 10.86% |
| Methyl Stearate (C 18:0) | 4.10 |
| Methyl Oleate (C18:1) | 25.91 |
| Methyl Linoleate (C18:2) | 52.99 |
| Methyl Linolenate (C18:3) | 6.14 |
| Others | traces |
| Total Saturates | 14.97 |
| Cloud Point: (C. °) | 3 |

FN. ¹ The carbon chain length, X, and number of carbon-carbon double bonds, Y, is indicated by the parenthetical item (CX:Y).

### EXAMPLE 9

24 g of soy methyl esters prepared according to Example 8 and 16.8 g of urea were added to 100 mL of methanol and the mixture was heated to 55 C.°, with constant stirring. A homogenous mixture was obtained with all the urea dissolving at this temperature. The mixture was then cooled in a water bath to 25° C. The urea clathrates were then separated by filtration. Methanol was recovered from the filtrate by flash evaporation. The filtrate was washed two times with equal volume of water (60 C.°, pH 3). 12.4 g of fractionated soy methyl ester (51.67% by wt of the starting soy methyl ester) was recovered with the composition and properties according to Table 10.

TABLE 10
Fractionated Soy Methyl Ester

| Fatty Acid Methyl Ester | Percentage by weight composition |
|---|---|
| Methyl Palmitate (C16:0) | 2.33 |
| Methyl Stearate (C18:0) | 0 |
| Methyl Oleate (C18:1) | 24.37 |
| Methyl Linoleate (C18:2) | 65.96 |
| Methyl Linolenate (C18:3) | 7.34 |
| Others (>C20) | traces |
| Total Saturates | 2.33 |
| Cloud Point: (C. °) | −23 |

### EXAMPLE 10

24. g of soy methyl ester prepared by example 8 and 24 g of urea were added to 100 mL of methanol. The mixture was heated to 55° C., with constant stirring. The homogenous mixture obtained was then cooled in a water bath to 25 to 20° C. The urea clathrates were separated by filtration. Methanol was removed from the filtrate by flash evaporation. The filtrate was washed two times with equal volume of water (60 C.°, pH 3). 10.32 g of fractionated soy methyl ester (42.92% by wt of the starting soy methyl ester) was recovered with the composition and properties according to Table 11.

TABLE 11
Fractionated Soy Methyl Ester

| | Percentage by weight |
|---|---|
| Fatty Acid Methyl Ester | composition |
| Methyl Palmitate (C16:0) | 0 |
| Methyl Stearate (C18:0) | 0 |
| Methyl Oleate (C18:1) | 19.19 |
| Methyl Linoleate (C18:2) | 72.34 |
| Methyl Linolenate (C18:3) | 8.46 |
| Others (>C20) | traces |
| Total Saturates | 0 |
| Cloud Point: (C. °) | −57 |

Applicants method as disclosed enables the fractionation of fatty acid methyl esters based on saturated vs unsaturated molecules from mixtures of saturated and unsaturated fatty acid methyl esters. Separated fractions may be achieved with the desired unsaturated fraction comprising from 15 to 0% by weight saturated fatty acid methyl esters, from 10 to 45% by weight monounsaturated fatty acid methyl esters, and from 50 to 85% polyunsaturated fatty acid methyl esters.

The C.P. of for mixtures of saturated and unsaturated fatty acid methyl esters may be reduced by preferably 10° C., more preferably 25° C., to 60° C. below the cloud point of the unfractionated fatty acid methyl ester mixture.

While the invention has been illustrated and described in detail in the foregoing description, such illustration and description is to be considered as exemplary and not restrictive in character, it being understood that only the preferred embodiments have been shown and described and that all changes and modifications that come within the spirit of the invention are desired to be protected.

What is claimed is:

1. A method for separating fractions of fatty acid methyl ester, comprising:
mixing the fatty acid methyl ester with an amount of urea and an amount of alcohol to make (i) a urea/fatty acid methyl ester ratio of from about 0.1:1 wt/wt to about 1:1 wt/wt and (ii) an alcohol/fatty acid methyl ester ratio of from about 3:1 wt/wt to about 10:1 wt/wt;
at a temperature at which a homogenous mixture is obtained;
cooling the fatty acid methyl ester/urea/alcohol mixture to a temperature where a solid phase and a liquid phase are formed; and
separating saturated fatty acid methyl ester enriched solid phase from the unsaturated fatty acid methyl ester enriched liquid phase.

2. The method of claim 1, wherein:
the alcohol is selected from the group consisting of methanol, ethanol, i-propanol, n-propanol, n-butanol, i-butanol, t-butanol, or a mixture thereof.

3. The method of claim 1, wherein:
where the alcohol is methanol.

4. The method of claim 1, wherein:
the fatty acid methyl ester/urea/alcohol mixture is cooled to a temperature from about 10 C.° to about 50 C.°.

5. The method of claim 1, wherein:
the solid phase is separated from the liquid phase by means of filtration, centrifugation, sedimentation, or decantation of the liquid phase.

6. The method of claim 5, wherein:
the solid phase is separated from the liquid phase by centrifugation.

7. A method of preparing enriched fatty acid methyl ester fractions comprising combining under reaction conditions a triglyceride fat and methanol;
the methanol being supplied in excess of the amount necessary to form a commercially complete methyl ester of fatty acid;
adding urea under conditions whereby upon mixing, the excess methanol enables formation of a homogeneous mixture of fatty acid methyl ester, urea, and methanol;
cooling the mixture to form urea clathrates;
physically separating the clathrates enriched in saturated fatty acid methyl ester from a liquid phase enriched in unsaturated fatty acid methyl ester.

8. The method of claim 7, wherein: the esterification of the fatty acid is commercially complete upon addition of urea.

9. The method of claim 7 wherein: the reaction conditions for combining methanol and triglyceride comprise a temperature of from 50 to 80° C.

10. The method of claim 9, wherein:
the cooled temperature is from 30 to 15° C.

11. Fatty acid methyl ester prepared according to claim 7.

12. Fatty acid methyl ester prepared according to claim 8.

13. A composition comprising the fatty acid methyl ester of claim 11 wherein the composition is selected from the group consisting of a fuel, a foodstuff, nutritive compositions, pharmaceuticals, cosmetics, dermatological compositions, coatings and paints.

14. A diesel engine or turbine engine fuel comprising an enriched fatty acid methyl ester prepared according to claim 1.

15. A diesel engine or turbine engine fuel comprising a fatty acid methyl ester of claim 11.

16. Fatty acid methyl ester mixtures wherein the fatty acid derived component comprises
(a) up to 5% by weight saturated hydrocarbons comprising from 16 to 18 carbon atoms;
(b) from 10 to 45% by weight monounsaturated hydrocarbons comprising 18 carbon atoms or more; and
(c) from 50 to 85% by weight of polyunsaturated hydrocarbons comprising 18 carbon atoms or more.

17. The fatty acid methyl ester mixture of claim 16 wherein the saturated hydrocarbons comprise not more than 2% by weight.
